SEQUENCE LISTING (26 sequences)

SEQ ID NO: 1 - protein, 30 aa, synthetic construct
  VARIANT 17: note = X can be A or P
  VARIANT 19: note = This position can be any amino acid
  VARIANT 20: note = X can be any amino acid
  VARIANT 20: note = X can be any amino acid
  VARIANT 20..21: note = These positions can be absent when position 19 is G
  VARIANT 24: note = X can be R or T
  VARIANT 28: note = X can be A, V, or T
  VARIANT 29: note = X can be A, V, or T
  VARIANT 1..4: note = These four positions may be present or absent as a unit
QEDHVQLVESGGGLVQXGGXXSLXLSCXXS

SEQ ID NO: 2 - protein, 25 aa, synthetic construct
  VARIANT 5: note = X can be V or L
  VARIANT 11: note = X can be L or V
  VARIANT 13: note = X can be Q or K
  VARIANT 16: note = X can be G or R
  VARIANT 19: note = X can be R or T
  VARIANT 23: note = X can be A, or V
  VARIANT 1: note = X can be E or Q
XVQLXESGGGXVXPGXSLXLSCXAS

SEQ ID NO: 3 - protein, 14 aa, synthetic construct
  VARIANT 9: note = X can be E, Q, or G
  VARIANT 12: note = X can be F, L, W, or G
  VARIANT 14: note = X can be A or S
  VARIANT 2: note = X can be F, Y, or V
WXRQAPGKXREXVX

SEQ ID NO: 4 - protein, 14 aa, synthetic construct
  VARIANT 14: note = X can be S or A
WVRQAPGKGLEWVX

SEQ ID NO: 5 - protein, 39 aa, synthetic construct
  VARIANT 26: note = X is S or N
  VARIANT 38: note = X is A or N
  VARIANT 39: note = X is A, T, or V
  VARIANT 20: note = X is V or L
YADSVKGRFTISRDNAKNTXYLQMNXLKPEDTAVYYCXX

SEQ ID NO: 6 - protein, 39 aa, synthetic construct
  VARIANT 19: note = X is T or S
  VARIANT 29: note = X is A or V
  VARIANT 39: note = X is R or K
  VARIANT 16: note = X is S or A
YADSVKGRFTISRDNXKNXLYLQMNSLRXEDTAVYYCXX

SEQ ID NO: 7 - protein, 10 aa, synthetic construct
WGQGTQVTVS

SEQ ID NO: 8 - protein, 10 aa, synthetic construct
  VARIANT 6: note = X can be L, T, or M
WGQGTXVTVS

SEQ ID NO: 9 - protein, 126 aa, synthetic construct
HVQLVESGGGLVQTGGSLRLSCAFSGYTFSTFPTAWFRQAPGKEREFVAGIRWNGGSRDYTEYADFVKGRFTISRDNAKNMIYLQMISLKPEDTALYYCAASHGVVDGTSVNGYRYWGQGTQVTVS

SEQ ID NO: 10 - protein, 118 aa, synthetic construct
HVQLVESGGGLVQAGGSLRLSCAASGSIFSSNAMSWYRQAPGKQRELVASITSGGNADYADSVKGRFTISRDKNTVYPEMSSLKPADTAVYYCHAVGQEASAYAPRAYWGQGTQVTVS

SEQ ID NO: 11 - protein, 125 aa, synthetic construct
DVQLVESGGGLVQAGGSLRLSCAASGFTFSNYVMYWGRQAPGKGREWVSGIDSDGSDTAYASSVKGRFTISRDNAKNTLYLQMNNLKPEDTALYYCVKSKDPYGSPWTRSEFDDYWGQGTQVTVS

SEQ ID NO: 12 - protein, 124 aa, synthetic construct
HVQLVESGGGLVQAGGSLTLTCAASGRTFSSETMDMGWFRQAPGKEREFVAADSWNDGSTYYADSVKGRFTISRDSAKNTLYLQMNSLKPEDTAVYYCAAETYSIYEKDDSWGYWGQGTQVTVS

SEQ ID NO: 13 - protein, 121 aa, synthetic construct
QVQLVESGGGLVQAGGSLRLSCAVSGRTFSIAGMGWFRQAPGKDREFLGGITWNDGTTWYADSVKGRFTISRDNAKNMLSLRMNSLKPEDTAVYYCAAGPRLGSTPRAYDYWGQGTQVTVS

SEQ ID NO: 14 - protein, 126 aa, synthetic construct
EVQLVESGGGLVQPGGSLRLSCAASGFTFSSYAMSWVRQAPGKGREWVAGITPGSGTFYADSVKGRFTISRDNAKNTLSLEINSLKPEDTALYYCAKCRQEFSWDFSSRDPDDFDYWGQGTQVTVS

SEQ ID NO: 15 - protein, 125 aa, synthetic construct
QVQLVESGGGLVQAGGSLRLSCAASGRTFSSYSMGWFRQAQGKEREFVATINGNGRDTYYTNSVKGRFTISRDDATNTVYLQMNSLKPEDTAIYYCAADKDVYYGYTSFPNEYEYWGQGTQVTVS

SEQ ID NO: 16 - protein, 125 aa, synthetic construct
HVQLVESGGGLVQAGGSLRLSCAVSGRTFSTYGMAWFRQAPGKERDFVATITRSGETTLYADSVKGRFTISRDNAKNTVYLQMNSLKIEDTAVYYCAVRRDSSWGYSRDLFEYDYWGQGTQVTVS

SEQ ID NO: 17 - protein, 86 aa, synthetic construct
EVQLVESGGGLVQPGGSLRLSCAASWYRQAPGKGREWVAYADSVKGRFTISRDKNTLYLQMNSLRAEDTAVYYCHAWGQGTLVTVS

SEQ ID NO: 18 - protein, 88 aa, synthetic construct
EVQLVESGGGLVQPGGSLRLSCAASWVRQAPGKGLEWVAYADSVKGRFTISRDNSKNTLYLQMNSLRAEDTAVYYCAKWGQGTLVTVS

SEQ ID NO: 19 - protein, 88 aa, synthetic construct
QVQLVESGGGLVQPGGSLRLSCAASWFRRAPGKEREFVAYADSVKGRFTISRDNAKNTLYLQMNSLRAEDTAVYYCAAWGQGTLVTVS

SEQ ID NO: 20 - protein, 116 aa, synthetic construct
EVQLVESGGGLVQPGGSLRLSCAASWVRQAPGKGLELVAYPDSVKGRFTISRDNAKNTLYLQMNSLRAEDTAVYYCASWGQGTLVTVSLQMNSLKAEDTAVYYCAAWGQGTLVTVS

SEQ ID NO: 21 - protein, 88 aa, synthetic construct
QVQLVESGGGLVQPGGSLRLSCAASWFRQAPGKGREFVSYADSVKGRFTISRDNAKNTLYLQMNSLKAEDTAVYYCAAWGQGTLVTVS

SEQ ID NO: 22 - protein, 88 aa, synthetic construct
QVQLVESGGGLVKPGGSLRLSCAASWFRQAPGKGLEWVSYADSVKGRFTISRDNAKNSLYLQMNSLRAEDTAVYYCARWGQGTLVTVS

SEQ ID NO: 23 - protein, 88 aa, synthetic construct
EVQLVESGGGLVQPGGSLRLSCAFSWFRQAPGKGREFVAYADFVKGRFTISRDNAKNTLYLQMNSLKAEDTALYYCAAWGQGTLVTVS

SEQ ID NO: 24 - protein, 88 aa, synthetic construct
EVQLVESGGGLVQPGGSLRLSCAASWVRQAPGKGREWVSYADSVKGRFTISRDNAKNTLYLQMNSLKAEDTALYYCVKWGQGTLVTVS

SEQ ID NO: 25 - protein, 88 aa, synthetic construct
EVQLVESGGGLVQPGGSLRLSCAASWVRQAPGKGLEWVSYADSVKGRFTISRDNAKNTLYLQMNSLRAEDTALYYCAKWGRGTLVTVS

SEQ ID NO: 26 - protein, 88 aa, synthetic construct
QVQLVESGGGLVQPGGSLRLSCAASWFRQAPGKEREFVAYADSVKGRFTISRDDAKNTLYLQMNSLKAEDTAVYYCAAWGQGTLVTVS

(19) United States
(12) Patent Application Publication
(10) Pub. No.: US 2025/0336476 A1
(43) Pub. Date: Oct. 30, 2025

(54) METHODS AND SYTEMS FOR NANOBODY HUMANIZATION

(71) Applicant: UNIVERSITY OF PITTSBURGH-OF THE COMMONWEALTH SYSTEM OF HIGHER EDUCATION, Pittsburgh, PA (US)

(72) Inventors: Zhe SANG, Pittsburgh, PA (US); Yi SHI, Wexford, PA (US)

(21) Appl. No.: 18/579,034

(22) PCT Filed: Jul. 12, 2022
(86) PCT No.: PCT/US2022/073637

§ 371 Date: Jan. 12, 2024

Related U.S. Application Data
(60) Provisional application No. 63/220,807, filed on Jul. 12, 2021.

(51) Int. Cl. G16B 30/10; G16B 15/00

(52) U.S. Cl. CPC G16B 30/10; G16B 15/00

(57) ABSTRACT

Disclosed are methods for humanizing antibodies and nanobodies and systems for performing the same.

This application claims the benefit of U.S. Provisional Application No. 63/220,807, filed on Jul. 12, 2021, which is incorporated herein by reference in its entirety.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Nov. 25, 2024, is entitled “10504-070US1_ST26.xml”, and is 36,750 bytes in size.

### I. BACKGROUND

V_{H}H antibodies or nanobodies (Nbs) have emerged as a compelling class of biologics. The first Nb drug (Cablivi) has recently been approved by the US Food and Drug Administration (FDA); more candidates are undergoing clinical trials. While these efforts have greatly inspired the innovative medical uses of Nbs and antibody fragments, there are remaining challenges for safe and effective applications to diseases in humans. In particular, anti-drug antibody (ADA) responses can reduce drug efficacy and, in rare cases, cause exacerbated inflammatory responses and toxicity. The underlying mechanism of ADA remains to be fully understood, and several factors including, critically, the use of non-human antibodies can contribute to the side effects. The consensus is that the humanization of xeno-species antibodies is necessary for drug development. Here “humanization” refers to increasing the similarity of antibodies of non-human origins to human antibodies. Thus far, efforts to humanize non-human antibodies has been best exemplified by the clinical benefits of humanizing murine antibodies, and humanized and fully human IgGs now dominate clinical development of biologicals.

Similar to the humanization of murine antibodies, strategies for Nb humanization are based on CDR grafting or FR resurfacing. One strategy involves 1) grafting antigen-specific CDRs to a specific human heavy chain variable domain (VHₕᵤₘₐₙ) framework, which often is a universal Nb framework. While this method has been successfully applied to some Nbs, using a single framework as the scaffold template may undermine the structural compatibility with many CDRs. While generally conserved, antibody frameworks nevertheless show substantial sequence and structural diversity to support infinite CDR loop conformations for antigen recognition. Such high scaffold diversity can not be fully represented by a small number of germline sequences. 2) Resurfacing uses available structures or structural models to guide the humanization of solvent-exposed frameworks, without changing buried residues. Resurfacing is based on the assumption that solvent-exposed, non-human residues do not contribute to the structural integrity and/or antigen engagement, which in most cases are likely invalid. In addition, unique CDR properties of Nbs, which remain to be fully investigated, can also contribute to ADA. Overall, there is a lack of systematic and structural investigations into Nb humanization, which is critical to moving therapeutic Nbs into clinical trials.

### II. SUMMARY

Disclosed are methods for humanizing antibodies and nanobodies and systems for performing the same.

In one aspect, disclosed herein are methods of humanizing nanobodies comprising a) matching nanobody sequences to human variable heavy chain (VHₕᵤₘₐₙ) sequences; b) performing a sequence alignment of the nanobodies and human variable chain sequences to identify framework sequence differences; c) performing intramolecular interaction analysis on nanobody structure measuring the structural stability to establish residues that are least likely to change the nanobody structure and more likely to be recognized by the human immune system; d) performing solvent accessibility analysis measuring to establish residues whose sidechains are exposed to solvent and most likely to be recognized by human immune system; and e) substituting residues that are not likely to have an impact on the structure, solubility, binding ability, but still exposed and likely to be recognized by human immune system. In some aspects, the method can further comprise performing nanobody structure prediction based on sequence and/or wherein when nanobody-antigen complex structure is available, the method can further comprise performing intermolecular contact analysis on the nanobody-antigen complex structure.

Also disclosed herein are methods of humanizing nanobodies of any preceding aspect, wherein the substitution comprises a substitution at framework region (FR) 1 (FR1) of the nanobody (such as, for example an alanine to proline substation at residue 14 (A14P) and/or an arginine to phenylalanine substitution at residue 27 (R27F)); wherein the substitution comprises a substitution at FR2 of the nanobody (such, as for example, a phenylalanine to valine substitution at residue 37 (F37V), tyrosine to valine (Y37V), a glutamate to glycine substitution at residue 44 (E44G), an arginine to leucine substitution at residue 45 (R45L), a phenylalanine to tryptophan substitution at residue 47 (F47W), and/or a leucine to tryptophan substitution at residue 47 (L47W)); wherein the substitution comprises a substitution at FR3 of the nanobody (such as, for example, a valine to leucine substitution at residue 75 (V75L), a tyrosine to arginine substitution at residue 83 (K83R), a proline to alanine substitution at residue 84 (P84A), an alanine to arginine substitution at residue 94 or an alanine to lysine substitution at residue 94(A94K)); and/or wherein the substitution comprises a substitution at FR4 of the nanobody (such as, for example, a glutamine to leucine substitution at residue 108 (Q108L)).

In one aspect, disclosed herein are methods of humanizing nanobodies of any preceding aspect, wherein measuring structural differences comprises measuring the distance between side chains and the antibody surface; wherein a residue with a distance of 3 Å or less is considered buried; wherein when a residue that is buried on a human antibody and exposed on a nanobody the residue is not substituted to humanize the nanobody.

Also disclosed herein are methods of humanizing nanobodies of any preceding aspect, wherein the intramolecular contact analysis measures interactions between FR2 and FR4 and/or interactions between FR2 and complementarity-determining region (CDR) 3 (CDR3). In one aspect, the intramolecular contact analysis measures specific intramolecular disulfide bonds present in nanobodies and not present in human variable heavy chains.

In one aspect, disclosed herein are methods of humanizing nanobodies of any preceding aspect, wherein the intermolecular contact analysis indicates direct involvement of residues in antigen binding; wherein residues directly involved in antigen binding are not substituted to humanize the nanobody.

Also disclosed herein are methods of humanizing nanobodies of any preceding aspect, wherein the process further comprises back translating humanized sequences into DNA sequences and synthesizing the sequence.

In one aspect, disclosed herein are methods of humanizing nanobodies of any preceding aspect, wherein humanization analysis steps b-d are performed using a system for automated structure guided nanobody humanization comprising a) nanobody structure modeling; b) sequence annotation; c) sequence analysis; d) structural analysis; and e) creating a humanization score.

Also disclosed herein are methods of humanizing nanobodies of any preceding aspect, wherein an indication of residue frequency of <10%, solvent exposure of residue, structural integrity as determined by intramolecular interaction analysis, and/or antigen binding as determined by intermolecular interaction analysis indicates a residue is selected for hybridization and wherein an indication of residue frequency of >10%, a buried residue, a lack of structural integrity as determined by intramolecular interaction analysis, and/or a lack of antigen binding as determined by intermolecular interaction analysis indicates a residue is excluded for hybridization.

In one aspect, disclosed herein are systems for automated structure guided nanobody humanization comprising a) nanobody structure modeling; b) sequence annotation; c) sequence analysis; d) structural analysis; and e) creating a humanization score. In one aspect, the system, further comprises intramolecular interaction analysis and/or intermolecular interaction analysis.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A, 1B, 1C, 1D, and 1E show analysis of T20 humanness scores for Nbs and VHs. Distributions of the T20 humanness scores based on specific sequences. FR: framework.

FIGS. 2A, 2B, 2C, 2D, and 2E show sequence conservation analysis of Nbs and VHₕᵤₘₐₙ. FIGS. 2A, 2B, 2C, and 2D show sequence logos of framework regions (FR1, FR2, FR3, and FR4) of Nbs and VHₕᵤₘₐₙ. In each panel, the top diagram shows the profile of Nbs, the second shows that of VHₕᵤₘₐₙ, the third shows the conserved residues specific to Nbs and the last, those specific to VHₕᵤₘₐₙ. FIG. 2A shows the analysis of FR1 for Nb (SEQ ID NO: 1), VHₕᵤₘₐₙ (SEQ ID NO: 2), Nb prevalent, and VHₕᵤₘₐₙ prevalent. FIG. 2B shows the alignment of FR2 for Nb (SEQ ID NO: 3), VHₕᵤₘₐₙ (SEQ ID NO: 4), Nb prevalent, and VHₕᵤₘₐₙ prevalent. FIG. 2C shows the alignment of FR3 for Nb (SEQ ID NO: 5), VHₕᵤₘₐₙ (SEQ ID NO: 6), Nb prevalent, and VHₕᵤₘₐₙ prevalent. FIG. 2D shows the alignment of FR4 for Nb (SEQ ID NO: 7), VHₕᵤₘₐₙ (SEQ ID NO: 8), Nb prevalent, and VHₕᵤₘₐₙ prevalent. FIG. 2E shows the percentage of conserved amino acid substitution on Nb FRs compared to VHₕᵤₘₐₙ.

FIGS. 3A, 3B, 3C, and 3D show analysis of buried residues in Nbs and VHₕᵤₘₐₙ. FIG. 3A shows distribution of depth of burial of residue side chain at the aligned position of the VHₕᵤₘₐₙ. Semi solvent-exposed FR2 residues L45 and W47 are highlighted. Error bars represent standard deviations. FIG. 3B shows that same as 3A for the aligned position of Nbs. Fully solvent-exposed FR2 residues R45 and F47 are highlighted. FIG. 3C shows ribbon diagram of the human antibody illustrating the extent of burial of FR2 residues (PDB: 6W41). The light chain is colored pink and the heavy chain is gray. Among 4 hallmark FR2 residues (37, 44, 45, and 477), only G44 is solvent-exposed. FIG. 3D shows Nb ribbon diagram showing burial of FR2 residues (PDB: 7JVB). Among 4 hallmark FR2 residues, E44, R45, and F47 are solvent-exposed.

FIGS. 4A, 4B, 4C, 4D, 4E, 4F, and 4G show conserved intramolecular interactions specific to Nbs. FIG. 4A shows upper panel: superimposition of 190 Nb structure backbone tracings. F/Y37 (FR2) is in yellow, R45 (FR2) in blue, and W103 (FR4) in salmon. Sidechains of the above residues are shown. Lower panel: the conserved interactions between FR2 and FR4 of a representative Nb (PDB:7JVB). FIG. 4B shows the distributions of distances and angles of the R45:W103 interactions based on the available Nb structures. FIG. 4C shows the distributions of distances and angles of the F/Y37:W103 interactions. FIG. 4D shows the percentage of contacts between 4 FR2 hallmark residues and CDR3 as a function of CDR3 length for Nbs and VHₕᵤₘₐₙ. FIG. 4E shows the percentage of CDR3s containing additional helix structure in different CDR3 length groups as contrasted to the distribution of CDR3 length for Nbs and VHₕᵤₘₐₙ. FIG. 4F shows a representative Nb structure showing interactions between a long CDR3 and two conserved FR2 residues (F37 and F47). FIG. 4G shows a representative Nb structure showing the presence of a short helicase structure in the CDR3 loop.

FIGS. 5A and 5B show the involvement of Nb FR2 to antigen binding. FIG. 5A shows heatmaps showing antigen contact propensity of human VHs and Nbs. Boxed area showed FR2 of Nbs involved more in antigen engagement. FIG. 5B shows a representative antigen Nb structure shows the interactions between V483,E484(RBD) and F47(Nb FR2).

FIG. 6 shows the schematic pipeline of Llamanade. The software is composed of five main modules: 1) structural modeling (purple), 2) sequence annotation (yellow), 3) sequence analysis (green), 4) structural analysis (pink), and 5) humanization score (brown). The input is Nb sequence information in a fasta format. Provided availability, additional structural input can be uploaded to facilitate the analysis. Nb sequences and structures will be first annotated by Llamanade to define CDRs and FRs. The sequence analysis module then selects candidate residues for humanization by comparing the sequence to the VHₕᵤₘₐₙ residue frequency matrix. The structural analysis including degree of burial, intra-and intermolecular interactions will be performed to evaluate the feasibility of humanization for candidate residues. The outputs are the humanized Nb sequence, structure and humanization score.

FIGS. 7A, 7B, 7C, and 7D show the humanization of SARS-CoV-2 Nbs by Llamanade and verification. FIGS. 7a shows structure model of binding of three classes of neutralizing Nbs to the RBD of SARS-CoV-2. The angiotensin-converting enzyme 2 (hACE2 in light yellow) is superimposed into the model. FIG. 7B shows a bar plot comparison of humanness scores (T20) of Nbs before and after humanization. FIG. 7C shows SDS PAGE gel picture showing the expression level of humanized Nbs. FIG. 7D shows the relative RBD binding affinities (nM) of wild type (WT) and humanized Nbs measured by ELISA.

FIGS. 8A, 8B, and 8C shows quantitative analysis of sequence conservation of Nbs and human VHs. FIG. 8A shows the crystallographic structure of a nanobody (PDB: 7JVB). Frameworks (FRs) and complementarity-determining regions (CDRs) are shown in different colors (FR1: salmon; CDR1: yellow; FR2: green; CDR2: cyan; FR3: blue; CDR3: purple; FR4: khaki). FIG. 8B shows a schematic of nanobody structure. FIG. 8C shows the conservation of the framework residue is calculated based on the entropy of amino acid variations in a given position in multiple alignments.

FIGS. 9A, 9B, 9C, 9D, and 9E show the benchmark of residue depth thresholds and analysis of conserved intramolecular interactions of Nbs. FIG. 9A shows average percentage of buried residue is plotted as a function of depth of buried residue side chain. FIGS. 9B and 9C show barplots showing the mean±SD (standard deviation) of the nearest atom distance for 9B) Nb residue pair R45:W103 and 9C) F/Y 37:W103. FIGS. 9D and 9E show barplots showing the number of Nbs (from a total of 190 Nbs) that have either the R45:W103 and the F/Y37:W103 interaction pairs

FIGS. 10A and 10B show VH-VL contacts in human IgGs and unique disulfide bond in Nbs. FIG. 10A shows a heatmap showing the VH-VL contact propensity of VHₕᵤₘₐₙ. FIG. 10B shows a representative Nb structure (PDB: 1ZMY) showing the disulfide bond formed between CDR3 and CDR1.

FIG. 11 shows sequence alignments of wild type and humanized Nbs. CDR sequences were highlighted. Humanized residues were in blue (prior) and red (after humanization). Shown are Nb9 (SEQ ID NO: 9), Nb17 (SEQ ID NO: 10), Nb34 (SEQ ID NO: 11), Nb36 (SEQ ID NO: 12), Nb64 (SEQ ID NO: 13), Nb93 (SEQ ID NO: 14), Nb95 (SEQ ID NO: 15), and Nb105 (SEQ ID NO: 16).

FIG. 12 shows framework sequence alignments of humanized Nbs to the best matched VHₕᵤₘₐₙ. Framework sequences of humanized Nbs are used to search the VHₕᵤₘₐₙ framework sequences with highest sequence identity. Shown are an alignment of humanized Nb17 (hNb17) (SEQ ID NO: 17) to AEX29471.1 (SEQ ID NO: 18), an alignment of humanized Nb21 (hNb21) (SEQ ID NO: 19) to CAX20928.1 (SEQ ID NO: 20), an alignment of humanized Nb64 (hNb64) (SEQ ID NO: 21) to AC596090.1 (SEQ ID NO: 22), an alignment of humanized Nb9 (hNb9) (SEQ ID NO: 23) to CAX20928.1 (SEQ ID NO: 20), an alignment of humanized Nb34 (hNb34) (SEQ ID NO: 24) to AC595693.1 (SEQ ID NO: 25), and an alignment of humanized Nb95 (hNb95) (SEQ ID NO: 26) to AC596090.1 (SEQ ID NO: 22).

FIG. 13 shows one-step purification of humanized SARS-CoV2-Nbs. Humanized Nbs were purified from E.coli whole cell lysis by using His6-cobalt resin. After imidazole elution, highly purified Nbs were analyzed by SDS-PAGE.

FIG. 14 shows RBD (SARS-CoV-2 spike) binding of wild-type and humanized Nbs by ELISA. The ELISA Optical Density readings at 450 nm were plotted against Nb concentrations (nM). hNb: humanized Nb.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Methods and Systems for Nanobody Humization

V_{H}H antibodies or nanobodies (Nbs) are small antigen-binding fragments that are derived from camelid (e.g., llama, alpaca, dromedary, and camel) heavy-chain antibodies. Nbs are composed of four conserved framework regions (FRs) that fold into β-sandwich core structures. Three hypervariable loops, or complementarity-determining regions (CDRs), are supported by the Nbs' robust fold to provide antigen-binding specificity. It has been shown that Nbs can preferentially target concave epitopes, efficiently interacting with target antigens using a smaller footprint. In many cases, the binding is markedly different from heterodimeric immunoglobulin G (IgG) antibodies, where the epitopes are generally more flat or convex.

The small size (˜15 kDa), robust fold, and lack of glycosylation enable rapid production of Nbs in microbes at low costs. Affinity matured Nbs are characterized by robust physicochemical properties including high solubility and stability, which are critical for drug development, production, transportation, and storage. Nbs are monomeric, which can be easily bioengineered into bispecific and multivalent modalities to enhance target binding and/or incorporate additional functionalities. Because of their small size, Nbs can bind compact molecular structures and penetrate tissues more efficiently than large IgG antibodies, thus facilitating molecular and diagnostic imaging applications.

In response to the COVID-19 (Coronavirus disease 2019) pandemic, thousands of highly potent and neutralizing Nbs have been developed by using in vivo affinity maturation coupled to a robust Nb drug discovery pipeline. These multiepitope Nbs highly specifically target the receptor-binding domain (RBD) of SARS-CoV-2 spike glycoprotein, and are cost-effective antiviral agents for the evolving virus. The outstanding preclinical efficacy and bioactivity of an inhalable construct have been recently demonstrated for inhalation therapy of SARS-CoV-2 infection by Nb aerosols. At an ultra-low dose, this innovative therapy has been shown to reduce lung viral titers by 6-logs to minimize lung pathology and prevent viral pneumonia. Moreover, high-resolution structure analyses have facilitated epitope mapping and classification of potent neutralizing Nbs into three main classes, which are characterized by distinct antiviral mechanisms. Systematic structural studies have provided insights into how Nbs uniquely target the spike to achieve ultrahigh-affinity binding and broadly neutralizing activities against the virus and its circulating variants.

Owing to these unique properties, Nbs have emerged as a compelling class of biologics. The first Nb drug (Cablivi) has recently been approved by the US Food and Drug Administration (FDA); more candidates are undergoing clinical trials. While these efforts have greatly inspired the innovative medical uses of Nbs and antibody fragments, there are remaining challenges for safe and effective applications to diseases in humans. In particular, anti-drug antibody (ADA) responses can reduce drug efficacy and, in rare cases, cause exacerbated inflammatory responses and toxicity. The underlying mechanism of ADA remains to be fully understood, and several factors including, critically, the use of non-human antibodies can contribute to the side effects. The consensus is that the humanization of xeno-species antibodies is necessary for drug development. Here “humanization” refers to increasing the similarity of antibodies of non-human origins to human antibodies. Thus far, efforts to humanize non-human antibodies has been best exemplified by the clinical benefits of humanizing murine antibodies, and humanized and fully human IgGs now dominate clinical development of biologicals.

Similar to the humanization of murine antibodies, strategies for Nb humanization are based on CDR grafting or FR resurfacing. One strategy involves 1) grafting antigen-specific CDRs to a specific human heavy chain variable domain (VHₕᵤₘₐₙ) framework, which often is a universal Nb framework. While this method has been successfully applied to some Nbs, using a single framework as the scaffold template may undermine the structural compatibility with many CDRs. While generally conserved, antibody frameworks nevertheless show substantial sequence and structural diversity to support infinite CDR loop conformations for antigen recognition. Such high scaffold diversity can not be fully represented by a small number of germline sequences. 2) Resurfacing uses available structures or structural models to guide the humanization of solvent-exposed frameworks, without changing buried residues. Resurfacing is based on the assumption that solvent-exposed, non-human residues do not contribute to the structural integrity and/or antigen engagement, which in most cases are likely invalid. In addition, unique CDR properties of Nbs, which remain to be fully investigated, can also contribute to ADA. Overall, there is a lack of systematic and structural investigations into Nb humanization, which is critical to moving therapeutic Nbs into clinical trials.

In this study, we have leveraged antibody/Nb next-generation sequencing (NGS) datasets and high-resolution structural data from the Protein Data Bank (PDB) to systematically analyze and compare Nbs, and mammalian (specifically, human and mouse) IgGs. Our analysis reveals the unique sequence and structural properties of Nbs and provides insights into Nb humanization. Guided by big data analysis, we have developed Llamanade—an open-source software to facilitate Nb humanization. Llamanade can rapidly optimize the solution and provide quantitative measurement of the extent of humanization. Finally, we have applied this tool to a cohort of structurally diverse and ultrapotent SARS-CoV-2 Nbs. Successfully humanized Nbs have demonstrated high bioactivities comparable to the non-humanized precursor Nbs.

In one aspect, disclosed herein are methods of humanizing nanobodies comprising a) matching nanobody sequences to human variable heavy chain (VHₕᵤₘₐₙ) sequences; b) performing a sequence alignment of the nanobodies and human variable chain sequences to identify framework sequence differences; c) performing intramolecular interaction analysis on nanobody structure measuring the structural stability to establish residues that are least likely to change the nanobody structure and more likely to be recognized by the human immune system; d) performing solvent accessibility analysis measuring to establish residues whose sidechains are exposed to solvent and most likely to be recognized by human immune system; and e) substituting residues that are not likely to have an impact on the structure, solubility, binding ability, but still exposed and likely to be recognized by human immune system. In some aspects, the method can further comprise performing nanobody structure prediction based on sequence and/or wherein when nanobody-antigen complex structure is available, the method can further comprise performing intermolecular contact analysis on the nanobody-antigen complex structure. As disclosed herein, an indication of residue frequency of <10%, solvent exposure of residue, structural integrity as determined by intramolecular interaction analysis, and/or antigen binding as determined by intermolecular interaction analysis indicates a residue is selected for hybridization and wherein an indication of residue frequency of >10%, a buried residue, a lack of structural integrity as determined by intramolecular interaction analysis, and/or a lack of antigen binding as determined by intermolecular interaction analysis indicates a residue is excluded for hybridization. In some aspects, the methods can further comprise back translating humanized sequences into DNA sequences and synthesizing the sequence.

It is understood and herein contemplated the substitutions for humanization can occur at any part of the nanobody including framework regions 1, 2, 3, and/or 4 (referred to herein as (FR1, FR2, FR2, and FR4, respectively). Accordingly in one aspect, disclosed herein are methods of humanizing nanobodies, wherein the substitution comprises a substitution at framework region (FR) 1 (FR1) of the nanobody (such as, for example an alanine to proline substation at residue 14 (A14P) and/or an arginine to phenylalanine substitution at residue 27 (R27F)); wherein the substitution comprises a substitution at FR2 of the nanobody (such, as for example, a phenylalanine to valine substitution at residue 37 (F37V), tyrosine to valine (Y37V), a glutamate to glycine substitution at residue 44 (E44G), an arginine to leucine substitution at residue 45 (R45L), a phenylalanine to tryptophan substitution at residue 47 (F47W), and/or a leucine to tryptophan substitution at residue 47 (L47W)); wherein the substitution comprises a substitution at FR3 of the nanobody (such as, for example, a valine to leucine substitution at residue 75 (V75L), a tyrosine to arginine substitution at residue 83 (K83R), a proline to alanine substitution at residue 84 (P84A), an alanine to arginine substitution at residue 94 or an alanine to lysine substitution at residue 94 (A94K)); and/or wherein the substitution comprises a substitution at FR4 of the nanobody (such as, for example, a glutamine to leucine substitution at residue 108 (Q108L)).

To minimize immunogenicity without perturbing the overall structure, we systematically analyzed high-resolution structures of antigen-antibody interactions to gain insights into the crucial, unique structural properties of Nbs. This structural information was used to differentiate buried residues from surface-exposed residues, which may elicit immunogenicity and therefore require humanization. Here, a residue is considered buried if the projecting side chain is 3 Å or more below the antibody surface. Accordingly, in one aspect, disclosed herein are methods of humanizing nanobodies, wherein measuring structural differences comprises measuring the distance between side chains and the antibody surface; wherein a residue with a distance of 3 Å or less is considered buried; wherein when a residue that is buried on a human antibody and exposed on a nanobody the residue is not substituted to humanize the nanobody. Additionally, large-scale structural analysis also reveals two type of intramolecular interactions that can contribute to structural integrity 1) interactions between FR2 and FR4 and 2) interactions between FR2 and CDR3. Thus, in one aspect, disclosed herein are methods of humanizing nanobodies, wherein the intramolecular contact analysis measures interactions between FR2 and FR4 and/or interactions between FR2 and complementarity-determining region (CDR) 3 (CDR3). In one aspect, the intramolecular contact analysis measures specific intramolecular disulfide bonds present in nanobodies and not present in human variable heavy chains.

Intermolecular contact analysis reveals direct involvement of specific FR1, FR2, FR3, and/or FR4 residues in antigen binding. In one aspect, disclosed herein are methods of humanizing nanobodies, wherein the intermolecular contact analysis indicates direct involvement of residues in antigen binding; wherein residues directly involved in antigen binding are not substituted to humanize the nanobody.

It is understood and herein contemplated that humanization analysis steps b-d (i.e., b) performing a sequence alignment of the nanobodies and human variable chain sequences to identify framework sequence differences; c) performing intramolecular interaction analysis measuring the structural differences to establish residues that are least likely to change the nanobody structure and more likely to be recognized by the human immune system; d) performing intermolecular contact analysis on the nanobody sequences) can be performed using a computer and software specifically designed for said analysis. In one aspect, disclosed herein are methods of humanizing nanobodies, wherein humanization analysis steps b-d are performed using a system for automated structure guided nanobody humanization comprising a) nanobody structure modeling; b) sequence annotation; c) sequence analysis; d) structural analysis; and e) creating a humanization score.

In one aspect, disclosed herein are systems for automated structure guided nanobody humanization comprising a) nanobody structure modeling; b) sequence annotation; c) sequence analysis; d) structural analysis; and e) creating a humanization score. In one aspect, the system, further comprises intramolecular interaction analysis and/or intermolecular interaction analysis. In one aspect, disclosed herein are the use of these systems to perform any of the humanization methods disclosed herein.

### 1. Antibodies

### (1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or fragments thereof” encompasses chimeric antibodies and hybrid antibodies, with dual or multiple antigen or epitope specificities, and fragments, such as F(ab′)2, Fab′, Fab, Fv, sFv, scFv, and the like, including hybrid fragments. Thus, fragments of the antibodies that retain the ability to bind their specific antigens are provided. For example, fragments of antibodies which maintain target binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

### (2) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an sFv, Fv, Fab, Fab′, F(ab′)2, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5, 939,598 (Kucherlapati et al.), U.S. Pat. No. 6,130,364 (Jakobovits et al.), and U.S. Pat. No. 6,180,377 (Morgan et al.).

### C. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Llamanade: An Open-Source Computational Pipeline for Robust Nanobody Humanization

### a) Results

### (1) Systematic and Quantitative Analysis of Nbs and IgG Antibodies

We compiled a high-quality Nb sequence library by randomly selecting 49,686 distinct, natural Nb sequences derived from previously generated NGS databases. Non-redundant human and murine IgG sequences (22,450 and 10,696, respectively) from the EMBL-Ig database were also selected to generate IgG sequence libraries. The variable region of antibody sequences and Nbs were numbered, and their CDRs and FRs were annotated using the Martin numbering scheme. To quantify the humanness level of Nbs, we used the T20 score, which has been shown to distinguish VHₕᵤₘₐₙ and VHₘₒᵤₛₑ with high specificity. The T20 score is calculated from the average sequence identities of the top 20 best-matched VHₕᵤₘₐₙ sequences in a large database (STAR Methods).

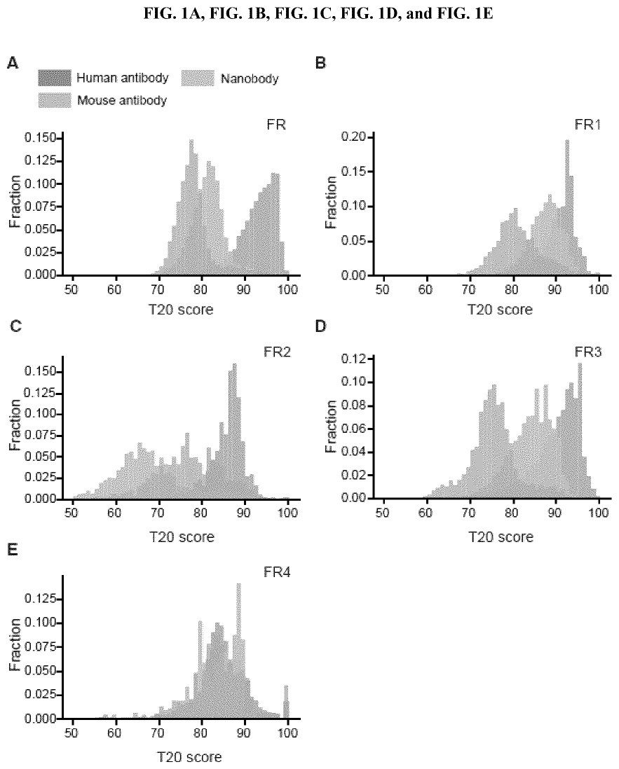

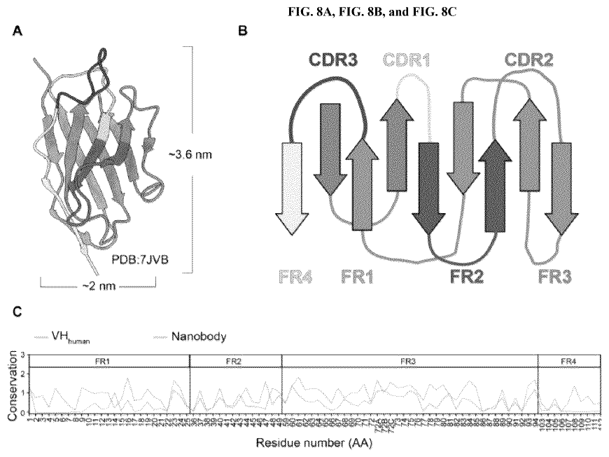

In addition, the T20 score can positively correlate with decreased immunogenicity of therapeutic antibodies to potentially inform clinical studies. An input Nb can be searched against our curated human IgG library to obtain the top 20 matched VHₕᵤₘₐₙₛ based on sequence identity. The percentage identity of the top 20 matches can be averaged to obtain the T20 score (STAR Methods). We calculated the T20 scores for the framework region (T20FR) of all the Nbs, VHₘₒᵤₛₑ, and VHₕᵤₘₐₙ in the respective libraries to generate the distribution plots (FIGS. 1A and 8). As expected, the scores of different antibody species largely follow normal distributions with VHₕᵤₘₐₙ slightly skewed toward the lower end. Compared to VHₘₒᵤₛₑ, Nbs are significantly more similar to VHₕᵤₘₐₙ, which is consistent with the previous analysis using a limited number of sequences. Notably, a substantial fraction of the Nbs is indistinguishable from VHₕᵤₘₐₙ based on overlapping T20 scores. The average T20FR values for Nbs and VHₘₒᵤₛₑ, are 79.7 and 72.1, respectively (FIG. 1A). High similarity between Nbs and VHₕᵤₘₐₙ sequences is primarily attributed to FR1, FR3, and FR4 among the four FRs (FIGS. 1B, 1D, and 1E). In contrast, the FR2s of Nbs are markedly different from their human counterparts (FIG. 1A). Moreover, while framework sequences are generally conserved, substantial variations are also evident (σₕᵤₘₐₙ VH=4.55, σₘₒᵤₛₑ VH=4.22, sNb =3.73). These structural variations underlie the potential challenges of using a single scaffold template to humanize a large repertoire of highly structurally diverse Nbs.

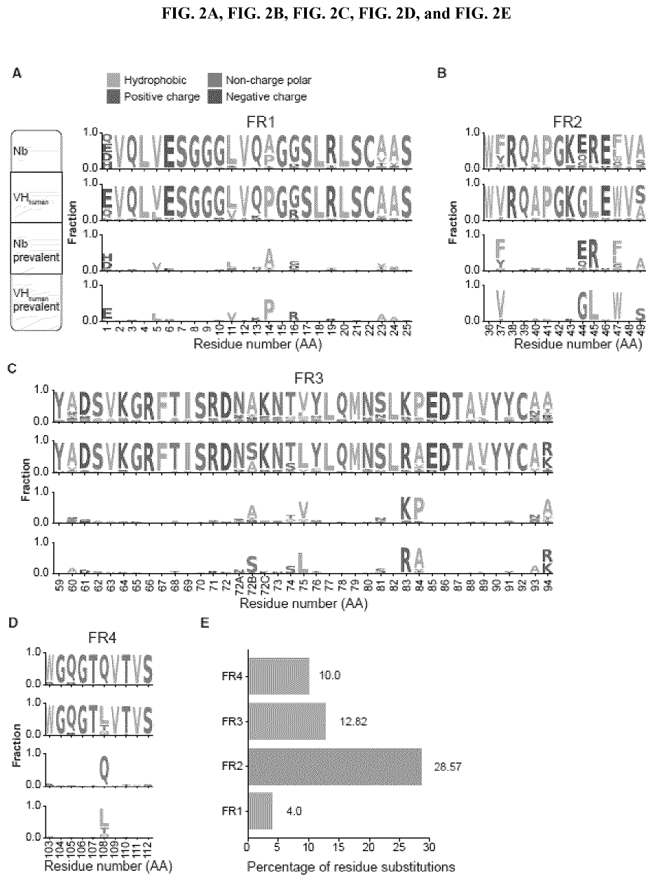

Identifying framework sequences that differ between Nbs and the top matched VHₕᵤₘₐₙ can inform humanization strategies. Nbs and the corresponding VHₕᵤₘₐₙ were aligned, and the resulting sequence logo profiles are presented in FIGS. 2A-2D (STAR Methods). The average percentages of residue substitution are 10%, 12.8%, 28.6%, and 4% for the respective four FRs (FIG. 2E). Hotspot (relatively abundant) residue substitutions (from Nbs to VHₕᵤₘₐₙ) were identified (STAR Methods) including FR1 residues: A14P, R27F; FR2 residues: F/Y37V, E44G, R45L, F/L47W; FR3 residues: V75L, K83R, P84A, A94R/K; and FR4 residue: Q108L. While the substitutions on three FRs share similar physicochemical properties, FR2 substitutions, especially F/Y37, E44, and R45, can reduce local hydrophilicity of Nbs. Large-scale sequence analysis reveals key differences between Nbs and VHₕᵤₘₐₙ, which can be modified to achieve higher similarity to human antibodies.

### (2) Large-Scale Structural Analysis Reveals Residue Interactions Specific to Nbs

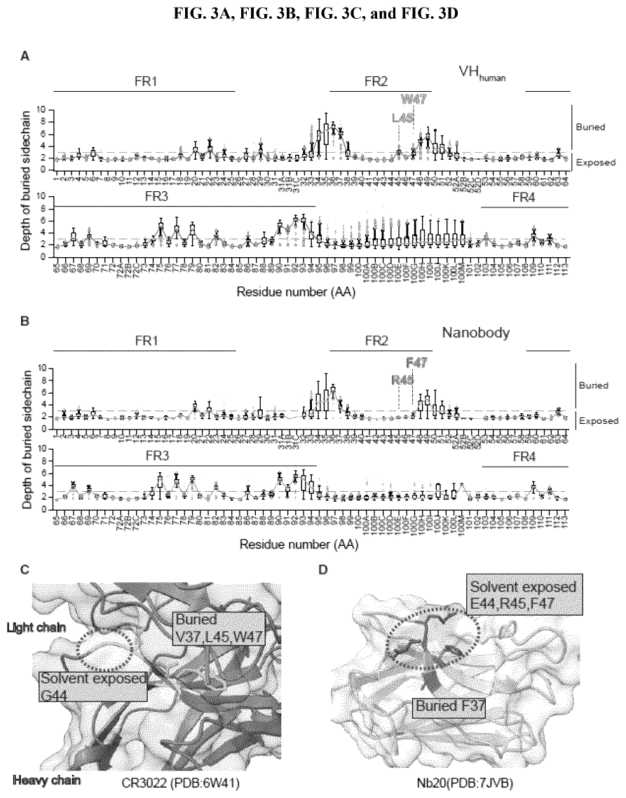

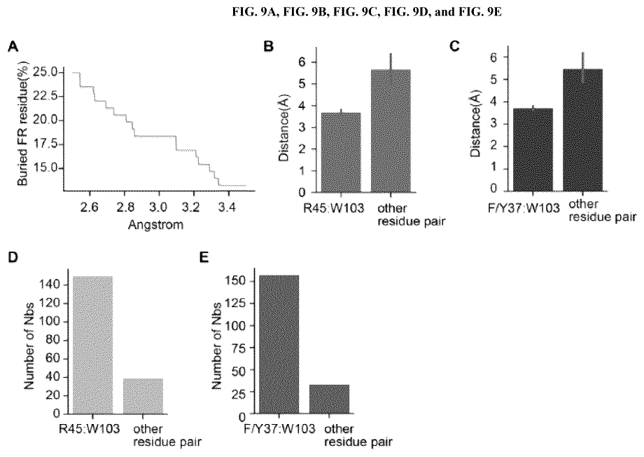

Given the structural differences between Nbs and VHₕᵤₘₐₙ, indiscriminate substitution of camelid Nb residues for human VHₕᵤₘₐₙ residues can result in perturbations to the overall Nb structure. In addition, surface-exposed residues are more likely to be recognized by the host immune response and are thus of particular interest for humanization. To minimize immunogenicity without perturbing the overall structure, we systematically analyzed high-resolution structures of antigen-antibody interactions to gain insights into the crucial, unique structural properties of Nbs. We assessed the structures of 190 distinct Nbs and 621 human IgGs from the AbBank. This structural information was used to differentiate buried residues from surface-exposed residues, which can elicit immunogenicity and therefore require humanization. Here, a residue is considered buried if the projecting side chain is 3 Å or more below the antibody surface (STAR Methods). As expected, a small fraction of antibody FR residues are buried, whereas the majority are solvent-exposed. Moreover, compared to VHₕᵤₘₐₙ (average 73%, σ=6.2%, or 24.42 buried residues), significantly more Nb FR residues (average 76.9%, σ=6.3%, or 18.18 buried residues) are solvent-exposed (FIGS. 3A and 3B). The major differences occur in the FR2, where R45 and F/L47 (Nbs) are fully exposed, whereas the corresponding human residues are buried (FIGS. 3C and 3D). These solvent-exposed residues in FR2 are likely to be important for the high solubility of Nbs and are not recommended for humanization. To substantiate this analysis, we varied the cutoff criteria in defining buried residues (from 2.5 to 3.5 Å) and found a small difference (12%) in those definitions (FIG. 9A). Since most FR residues are highly conserved between VHₕᵤₘₐₙ and Nbs and on average ten FR residues per Nb are needed for humanization, a 12% difference here corresponds to only one residue uncertainty and therefore may not significantly impact the decision for Nb humanization.

Large-scale structural analysis also reveals two types of intramolecular interactions that can contribute to structural integrity. The first is between FR2 and FR4. Here, W103 (FR4) can associate with R45 (FR2) by a conserved cation-p interaction, which is replaced by hydrophobic interaction of L45-W103 in VHₕᵤₘₐₙ. In addition, W103 can also interact with F/Y37 using a π-π interaction, which is absent in the IgG VH domains. To substantiate these observations, we measured the distances and angles of the charged atoms of R45 with respect to the centroids of aromatic rings from W103. We also measured those between F/Y37 and W103 (STAR Methods). The average distance and angle of the R45-W103 and F/Y37-W103 interactions are 3.9 Å/65.7° and 4.1 Å /31°, supporting the respective bond formations (cation-π and π-π interactions). We found that R45-W103 and F/Y37-W103 interactions are highly conserved (79.0% and 82.6% in the PDB structures, respectively) (FIGS. 9B-9E). These interactions are likely critical to support the robust scaffold and excellent physicochemical properties of Nbs.

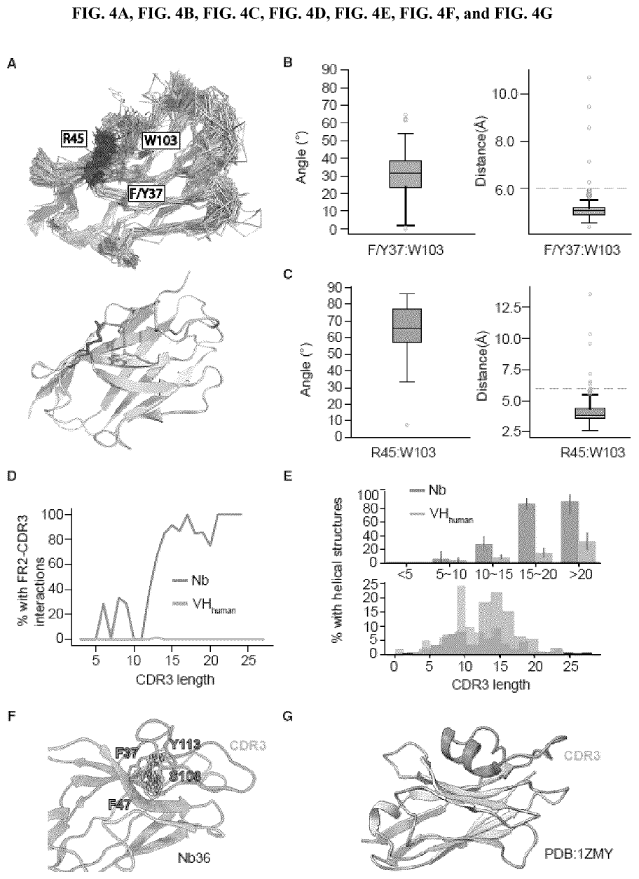

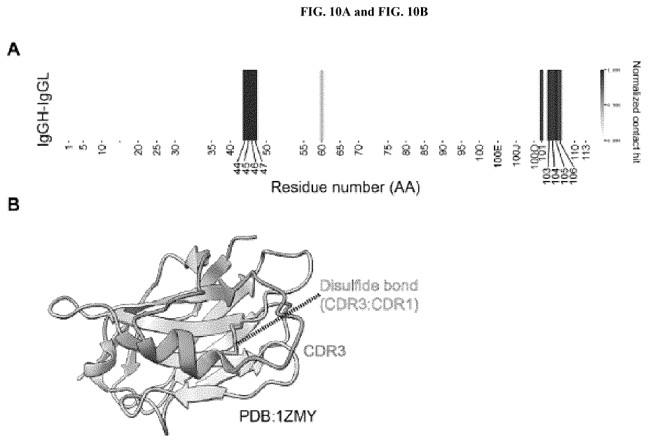

The second important intramolecular interaction specific to Nbs is between the FR2 and CDR3 (FIG. 4D). The frequency of this bond positively correlates with CDR3 length. Over 80% of long CDR3s (>15 residues) were found to have this interaction (FIG. 4D). In addition, most long CDR3s contain a small helix structure that was rarely identified on VHₕᵤₘₐₙ (FIGS. 4E and 4G). Analysis reveals that the long CDR3 loops of Nbs can fold back to interact with two specific FR2 residues (positions 37 and 47; FIG. 4F), where the corresponding VHₕᵤₘₐₙ residues are used to interact with the light chains (VLs) (FIG. 10A). These specific FR2-CDR3 interactions may impact humanization in two ways: (1) the long CDR3s can shield potentially immunogenic residues on FR2, which are otherwise solvent-exposed. (2) The helix associated with the CDR3 loops can potentially enhance immunogenicity and the development of ADA in clinical applications (FIG. 4G). Nevertheless, these FR2 residues unique to Nbs must be conserved, as resurfacing these residues can alter CDR3 conformations and compromise antigen binding.

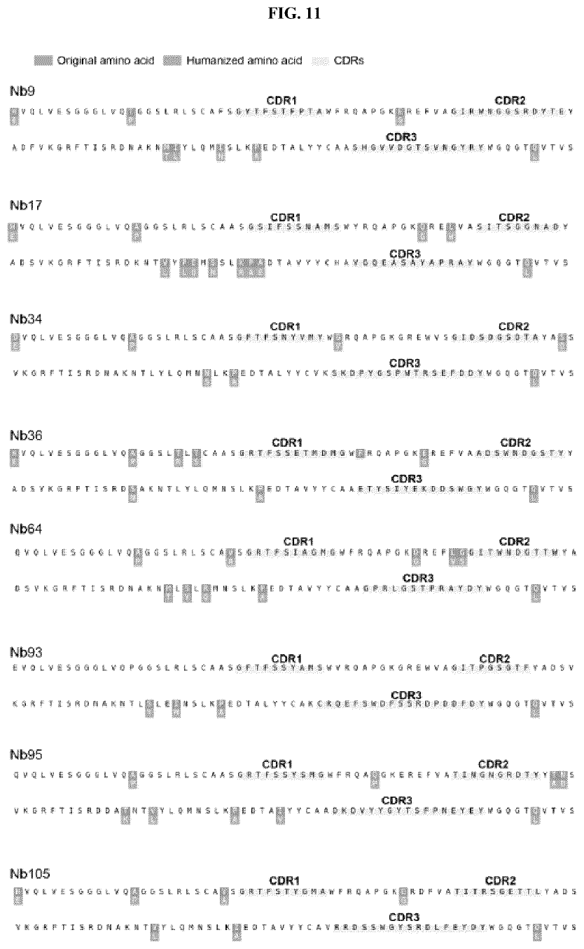

Moreover, specific intramolecular disulfide bonds (between CDR3 and CDR1/2) were identified in approximately 10% of Nbs that we have analyzed but were absent in VHₕᵤₘₐₙ (FIG. 11B). The disulfide bridges (FIG. 10B) are exclusively buried for folding, and the corresponding residues are not considered for humanization in our software.

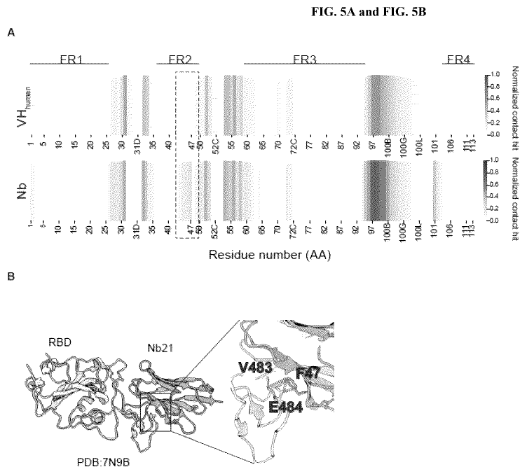

Finally, intermolecular contact analysis reveals direct involvement of specific FR2 residues (44,45 and 47) in antigen binding (FIG. 5). Based on the antigen-Nb structures, approximately 20% of Nbs use these FR2 residues for antigen binding. In addition, it appears in vivo affinity-matured Nbs use them less extensively for binding compared to in vitro selected Nbs. Without detailed structure information of antigen-Nb interactions, these FR2 residues can be ideally excluded from humanization to better preserve structural integrity.

### (3) Development of an Open-Source Software to Facilitate Rational Nb Humanization

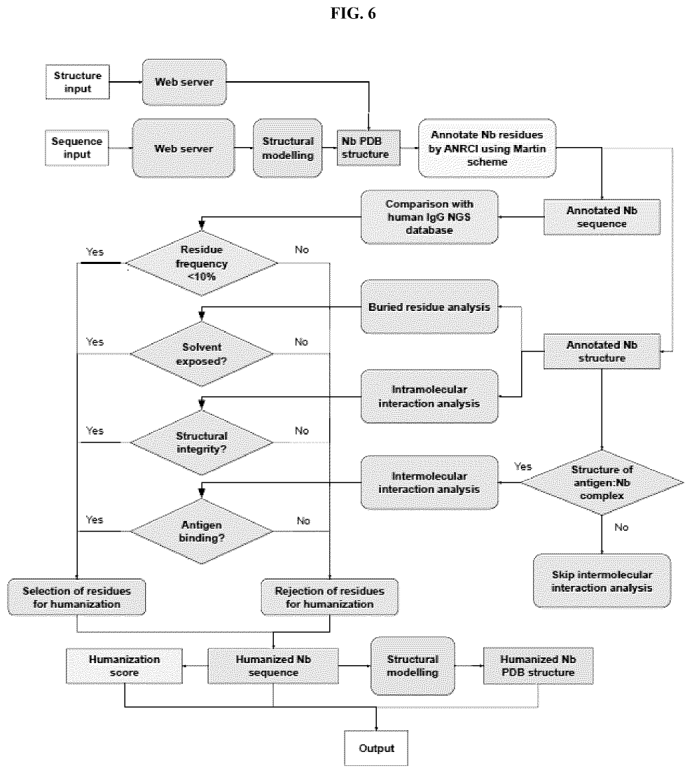

Based on big data analysis, we developed an open-source software to facilitate automated, structure-guided, and robust Nb humanization. Llamanade is composed of five main modules (FIG. 6): (1) Nb structural modeling, (2) sequence annotation, (3) sequence analysis, (4) structural analysis, and (5) humanization score. The input is a Nb sequence in a simple fasta format. The structural model is generated by Modeller or NanoNet (STAR Methods). Alternatively, the input can be high-resolution structures of Nbs or antigen-Nb complexes. Nb sequence can be annotated using the Martin scheme to define FRs and CDRs. To select the best VHₕᵤₘₐₙ templates for Nb humanization, we first use Nb sequences (from the NGS database) to fetch the top 20 VHₕᵤₘₐₙ in our human IgG library that are highly similar to Nbs based on T20_{FR} score (FIG. 2, STAR Methods). 6,070 VHs were obtained after this analysis. The sequences are aligned to calculate the amino acid frequency at each position to generate a position probability matrix. Annotated input Nbs can be compared to this matrix to choose FR residues for humanization. Candidate residues can be selected if their occurrences are sufficiently low (,10%) at the corresponding position of VHₕᵤₘₐₙ. Substitution of these residues can result in incompatibility of these rare VHₕᵤₘₐₙ frameworks with specific CDR loop conformations that compromise the structural and physicochemical properties of the Nbs after humanization.

In parallel, our software can perform structural analysis to determine if the candidate Nb residues are solvent exposed or not. Only solvent-exposed residues that do not participate in backbone intramolecular interactions (structural integrity) and do not impact antigen-Nb interactions (FIG. 4) can be humanized. In particular, specific FR2 residues at positions 37, 45, and 47 are not recommended to change because of their involvement in either backbone interactions or antigen binding (FIGS. 3 and 4). A humanization score can be calculated based on T20_{FR} to quantify the humanness level of Nbs (FIG. 1). The output files include the annotated humanized Nb sequence, modeled structure, and the humanization score. Llamanade is user-friendly and the full analysis takes less than 1 min/Nb to complete on a local device. In addition, the service can be deployed to a webserver to provide better accessibility and visualization to the community.

### (4) Humanization of Ultrapotent SARS-CoV-2 Neutralizing Nbs by Llamanade

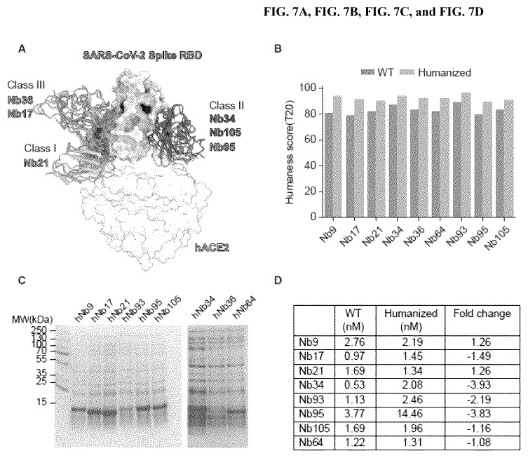

To demonstrate the robustness of Llamanade, we humanized nine highly potent SARS-CoV-2 neutralizing Nbs that have been recently identified. These Nbs target the RBD of the virus spike and are structurally diverse, falling into three main epitope classes (FIG. 7A). Class I Nbs include Nb 21 and presumably Nb64. They can directly block the ACE2 receptor-binding sites of RBD to potently neutralize SARS-CoV-2 at as low as ˜0.3 ng/ml (22 pM), which is unprecedented for antiviral antibody fragments. Class II Nbs (34, 95, 105, and presumably 93) strongly bind conserved RBD epitopes and neutralize the virus at 30 ng/ml. Class III Nbs (17, 36, and presumably 9) recognize relatively conserved sites including cryptic neutralizing epitopes where large human antibodies may not be able to access. Here robust humanization can help preclinical and clinical development of antiviral Nbs.

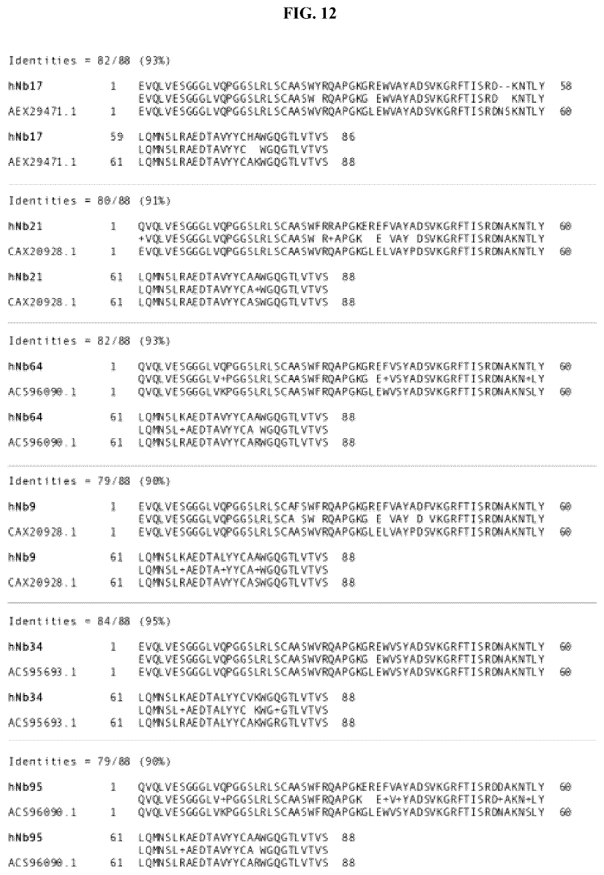

Using sequence as input, these RBD Nbs were humanized by Llamanade, which significantly improved the humanness level of Nb frameworks. The median T20 score was substantially increased from 82.8 (before humanization) to 92.4 (after humanization), which corresponds to the humanization of seven FR residues (FIGS. 7B and 11). The median framework identity of these highly humanized Nbs to the top matched VH sequence ranges between 90% and 95% (FIG. 12).

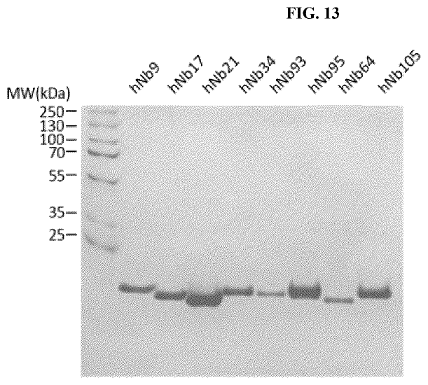

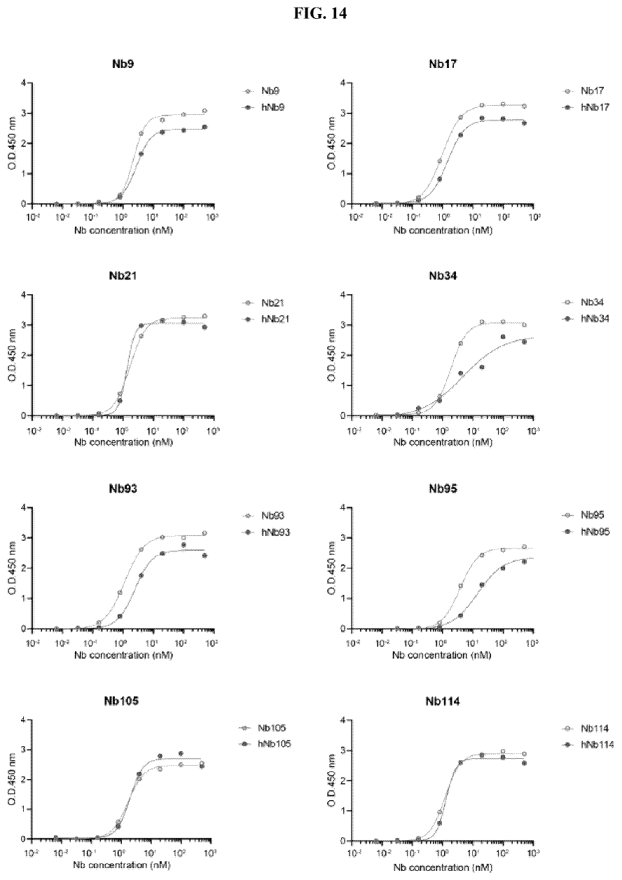

After humanization, Nbs were back translated into DNA sequences, which were synthesized in vitro and cloned into an expression vector (pET-21b) for recombinant protein productions in E. coli (STAR Methods). Eight of nine humanized Nbs were readily expressed in the whole-cell lysis (FIG. 7C) comparable to their native forms. They were one-step purified by His-cobalt resin with excellent solubility and yield (STAR Methods; FIG. 13). The only exception was Nb36. While a fraction of hNb36 can be purified from the cell lysis (FIG. 13), the majority was found in the inclusion body and was excluded for enzyme-linked immunosorbent assay (ELISA) analysis due to potentially inferior solubility. To assess the bioactivities of these humanized Nbs, we performed the ELISA and confirmed the comparably high activities (±4-fold) to the non-humanized precursors (FIGS. 7D and 14). These highly humanized ultrapotent SARS-CoV-2 Nbs recapitulated robust physicochemical and structural properties that are critical for inexpensive manufacturing toward clinical development.

### b) Discussion

Nbs are characterized by small size, high solubility, and stability for advanced biomedical uses. Technology advancement has recently enabled rapid isolation of thousands of sub-nM affinity and multi-epitope Nbs for specific antigen binding, which opens exciting possibilities for drug development. Recently, ultrapotent Nbs have shown great promise as costeffective antiviral agents to help curve the pandemic caused by SARS-CoV-2. Stable and ultrapotent Nbs can be inhale delivered by aerosolization with high bioavailability to treat pulmonary infections efficiently. Humanization has been considered as a key step for therapeutic development of xeno-species antibodies including Nbs of camelid origin. However, systematic investigations into Nb humanization based on large-scale sequence and structural analyses remain unavailable. To our knowledge, there is no dedicated software available for Nb humanization. To fill this gap, we have developed the open-source Llamanade to facilitate robust and fast Nb humanization. Using Llamanade, we quickly humanized a cohort of structurally diverse and ultrapotent SARS-CoV-2 neutralizing Nbs. Critically, these highly humanized Nbs demonstrated robust physicochemical and structural properties and high bioactivities comparable to the natural, affinity-matured precursors.

The underlying mechanisms of ADA remain to be investigated. However, we note that multiple factors, beyond the use of non-human antibodies, may contribute to the ADA responses. These include (1) impurity of antibodies, (2) the route and dose of administration, and (3) solubility of antibody or antigen-antibody complex. Inspired by the FDA approval of the first Nb therapy, a number of preclinical and clinical programs have been initiated, providing critical insights into the safety profiles, dosing regime, and efficacy of therapeutic Nbs. For example, recent studies based on dozens of preclinical results found that ADA responses were minor and were generally non-neutralizing to compromise efficacy. Clinical trials have further revealed limited safety issues of Nbs (mostly humanized Nbs) in both patients and healthy volunteers. Nevertheless, ADA has been detected in both preclinical and clinical development. For example, a humanized bivalent anti-cancer Nb targeting human cytokine receptor (hIL-6R) was associated with adverse effects during clinical trials and was terminated prematurely. Potential immunogenicity and adverse effects of a non-humanized HER2 Nb were also detected in phase I trial, which showed moderate increase over the course of the trial. These studies underscore the requirement of comprehensive evaluations of ADA and toxicology studies before moving Nbs into clinical trials. Here, the development of robust humanization methods can help improve the translational potentials of therapeutic Nbs.

In summary, we have developed a robust, fast, and user friendly tool to facilitate automated Nb humanization. Llamanade is the first dedicated software for Nb humanization. It is freely accessible and can be extended for the analysis of other VH-like scaffolds such as shark single-domain antibodies (VNAR). In a parallel effort, NanoNet was developed, which employs machine learning for accurate and high-throughput modeling of Nb structures. These tools have been integrated to further advance Nb-based biomedical research and therapeutic development. The source codes and web server are freely available.

### c) Data and Code Availability

All original code has been deposited at Github and is publicly available. DOIs are listed in the Key resources table. Any additional information required to reanalyze the data reported in this paper is available from the lead contact upon request.

### d) Method Details

### (1) Sequence Dataset

VHₕᵤₘₐₙ and VHₘₒᵤₛₑ sequences were downloaded from EMBL-Ig. Data were filtered by removal of duplication and incomplete VH sequences, which leads to a final of 22,450 and 10,696 non-redundant and high quality VH sequences. 49,686 distinct Nb sequences derived from previously generated NGS databases and 14,348 share less than 90% sequence identity based on clustering result from CD-HIT.

### (2) Structural Dataset

1694 antigen: IgG complex structures and 246 antigen: Nb complex structures were obtained from AbBank. Duplicated sequences were removed from the dataset resulting in 621 distinct IgG and 190 distinct Nb. Structures were annotated based on the Martin scheme. In addition, 7 SARS-CoV-2 RBD:Nb structures were obtained from a recent study by Dapeng et al.

### (3) Humanness T20 Score

The T20 scoring system was implemented according to the method described in. In this study, 22,450 non-redundant human VH sequences were used to build the BLAST reference database. To obtain the T20 score of a given VH/VHH sequence, the query sequence is searched against the reference database by BLASTP. For an input VHₕᵤₘₐₙ, the top 20 matched sequences are retrieved and corresponding sequence identities are averaged to calculate the T20 score. For a VH sequence from any other species, the top 20 matched sequences can be retrieved and used to calculate T20 score. In addition to a full sequence-based T20 scorer, we also implemented a FR based T20 scorer. More specifically, framework sequences of human VHs were extracted to construct the reference database and the framework region of the query sequence was used for the search. For each FR based T20 score, we first extracted FR sequences from the human VH sequence databases to build FR-based BLAST reference databases. Next, we calculated a T20 (T20_{FR}1-4) based on the input FR sequence and reference FR sequence database.

### (4) Antibody Alignment and Numbering

To identify FRs and CDRs, antibodies in the sequence and structural dataset were aligned by ANARCI using the Martin numbering scheme. The Martin numbering scheme assigns a number to each amino acid indicating the topological position in structure. After alignment, aligned positions with more than 99% of introduced gap were removed. The frequencies of amino acids at each aligned position were calculated from multiple sequence alignment to generate position-probability-matrix (PPM). To visualize prevalent residues, the Nb PPM was subtracted by VHₕᵤₘₐₙ. After subtraction, positive values in the matrix indicate specific residues are more prevalent in Nbs, whereas negative values indicate more prevalent in VHₕᵤₘₐₙ. Residues with a subtracted value of >0.1 were considered hotspot residues.

### (5) Structural Analysis of Buried Residues

The degree of burial for a residue is quantified by measuring the depth of the side chain below the protein surface using the ResidueDepth module in BioPython. The depth of the residue side chain is calculated by average distance to surface for all atoms. The protein surface was generated by software MSMS. A cutoff of 3.03 Å defining the state of burial or exposure to solvent was used. This analysis was performed in the absence of antigens.

### (6) Contact Analysis of Antigen-Antibody Interactions

The Ca distance between two residues was calculated for every antibody-antigen residue pair using ProDy. A residue pair with Ca distance less than 8 angstrom was considered for interaction.

### (7) Analysis of Nanobody Intramolecular Interaction

Interactions such the salt bridge, cation-π, π-π were investigated. The following criteria were used to predict the presence of a certain interaction:

Salt bridge: The distance between two opposite charge atoms between two residues is less than 4 angstrom.

Cation-π: The distance between the centroid of the aromatic ring from TRP/PHE/TYR and the charged atom from LYS/ARG is less than 6 angstrom.

π-π: The distance between centroids of two aromatic rings from TRP/PHE/TYR is within 4˜6 angstrom.

### (8) Structural Modeling

### (a) Structural Template Selection

Using the Nb sequence as input, sequences from a dataset comprising 190 distinct Nb structures can be aligned to the input sequence. The Nb structure with highest sequence identity to the input Nb sequence can be selected as the template structure for modeling.

### (b) Modeling by MODELLER

MODELLER automatically calculates 20 comparative Nb structure models using input Nb sequence, the template Nb structure and sequence alignment information between the input and template Nb sequences. The Discrete optimized protein energy (DOPE) score is specified as objective function in MODELLER to evaluate quality of model. We rank 20 models based on the DOPE score and only the model with the best DOPE score (i.e. lowest energy) is selected for downstream structural analysis.

### (9) Modeling by NanoNet

An deep learning model based end-to-end Nb structure modeling software NanoNet has been integrated into Llamanade for structural modeling of Nbs. Provided a Nb sequence, NanoNet can rapidly output all the Ca 3D coordinates of the Nb directly and without structural templates. After obtaining the backbone structure of Nb, amino acid side chains are constructed by PULCHRA.

### (10) ELISA (Enzyme-Linked Immunosorbent Assay)

Antigens (RBD) were coated onto 96-well ELISA plates, with 150 ng of protein per well in the coating buffer (15 mM sodium carbonate, 35 mM Sodium Bicarbonate, pH 9.6) at 4° C. overnight. The plates were decanted, washed with a buffer (1×PBS, 0.05% Tween 20), and blocked for 2 hours at room temperature (1×PBS, 0.05% Tween 20, 5% milk powder). Nanobodies were serially diluted by 5-fold in blocking buffers. Anti-T7 tag HRP-conjugated secondary antibodies were diluted at 1:5000 and incubated at room temperature for 1 hour. Upon washing, samples were further incubated in the dark for 10 minutes with freshly prepared 3,30, 5,50-Tetramethylbenzidine (TMB) substrate. Upon quenching the reaction with a STOP solution, the plates were measured at wavelengths of 450 nm with background subtraction at 550 nm. The raw data were processed and fitted into the 4PL curve using the Prism Graphpad 9.0. IC50s were calculated and fold changes of binding affinity were calculated to generate the heatmap.

### D. REFERENCES

Abhinandan, K. R., and Martin, A. C. (2008). Analysis and improvements to Kabat and structurally correct numbering of antibody variable domains. Mol. Immunol. 45, 3832-3839.
Ackaert, C., Smiejkowska, N., Xavier, C., Sterckx, Y. G. J., Denies, S., Stijlemans, B., Elkrim, Y., Devoogdt, N., Caveliers, V., Lahoutte, T., et al. (2021). Immunogenicity risk profile of nanobodies. Front. Immunol. 12, 632687.
Bakan, A., Dutta, A., Mao, W., Liu, Y., Chennubhotla, C., Lezon, T. R., and Bahar, I. (2014). Evol and ProDy for bridging protein sequence evolution and structural dynamics. Bioinformatics 30, 2681-2683.
Bao, G., Tang, M., Zhao, J., and Zhu, X. (2021). Nanobody: a promising toolkit for molecular imaging and disease therapy. EJNMMI Res. 11, 6.
Camacho, C., Coulouris, G., Avagyan, V., Ma, N., Papadopoulos, J., Bealer, K., and Madden, T. L. (2009). BLAST+: architecture and applications. BMC Bioinf. 10, 421.
Cohen, T., Halfon, M., and Schneidman-Duhovny, D. (2021). NanoNet: rapid end-to-end nanobody modeling by deep learning at sub angstrom resolution. bioRxiv.
Dalkas, G. A., Teheux, F., Kwasigroch, J. M., and Rooman, M. (2014). Cation-pi, amino-pi, pi-pi, and H-bond interactions stabilize antigen-antibody interfaces. Proteins 82, 1734-1746.
Desmyter, A., Transue, T. R., Ghahroudi, M. A., Thi, M. H., Poortmans, F., Hamers, R., Muyldermans, S., and Wyns, L. (1996). Crystal structure of a camel single-domain VH antibody fragment in complex with lysozyme. Nat. Struct. Biol. 3, 803-811.
Dougherty, D. A. (2013). The cation-pi interaction. Acc. Chem. Res. 46, 885-893.
Duggan, S. (2018). Caplacizumab: first global approval. Drugs 78, 1639-1642.
Dunbar, J., and Deane, C. M. (2016). ANARCI: antigen receptor numbering and receptor classification. Bioinformatics 32, 298-300.
Gao, S. H., Huang, K., Tu, H., and Adler, A. S. (2013). Monoclonal antibody humanness score and its applications. BMC Biotechnol. 13, 55.
Greenberg, A. S., Avila, D., Hughes, M., Hughes, A., Mckinney, E. C., and Flajnik, M. F. (1995). A new antigen receptor gene family that undergoes rearrangement and extensive somatic diversification in sharks. Nature 374, 168-173.
Hamers-Casterman, C., Atarhouch, T., Muyldermans, S., Robinson, G., Hamers, C., Songa, E. B., Bendahman, N., and Hamers, R. (1993). Naturally occurring antibodies devoid of light chains. Nature 363, 446-448.
Hansel, T. T., Kropshofer, H., Singer, T., Mitchell, J. A., and George, A. J. (2010). The safety and side effects of monoclonal antibodies. Nat. Rev. Drug Discov. 9, 325-338.
Harding, F. A., Stickler, M. M., Razo, J., and DuBridge, R. B. (2010). The immunogenicity of humanized and fully human antibodies: residual immunogenicity resides in the CDR regions. MAbs 2, 256-265.
Jovcevska, I., and Muyldermans, S. (2020). The therapeutic potential of nanobodies. BioDrugs 34, 11-26.
Klarenbeek, A., El Mazouari, K., Desmyter, A., Blanchetot, C., Hultberg, A., de Jonge, N., Roovers, R. C., Cambillau, C., Spinelli, S., Del-Favero, J., et al. (2015). Camelid Ig V genes reveal significant human homology not seen in therapeutic target genes, providing for a powerful therapeutic antibody platform. MAbs 7, 693-706.
Li, W., and Godzik, A. (2006). Cd-hit: a fast program for clustering and comparing large sets of protein or nucleotide sequences. Bioinformatics 22, 1658-1659.
Lu, R. M., Hwang, Y. C., Liu, I. J., Lee, C. C., Tsai, H. Z., Li, H. J., and Wu, H. C. (2020). Development of therapeutic antibodies for the treatment of diseases. J. Biomed. Sci. 27, 1.
Masuda, K., Sakamoto, K., Kojima, M., Aburatani, T., Ueda, T., and Ueda, H. (2006). The role of interface framework residues in determining antibody V(H)/V(L) interaction strength and antigen-binding affinity. FEBS J. 273, 2184-2194.
Mitchell, L. S., and Colwell, L. J. (2018). Comparative analysis of nanobody sequence and structure data. Proteins 86, 697-706.
Morrison, C. (2019). Nanobody approval gives domain antibodies a boost. Nat. Rev. Drug Discov. 18, 485-487.
Nambulli, S., Xiang, Y., Tilston-Lunel, N. L., Rennick, L. J., Sang, Z., Klimstra, W. B., Reed, D. S., Crossland, N. A., Shi, Y., and Duprex, W. P. (2021). Inhalable Nanobody (PiN-21) prevents and treats SARS-CoV-2 infections in Syrian hamsters at ultra-low doses. Sci. Adv. 7, eabh0319.
Rossotti, M. A., Be'langer, K., Henry, K. A., and Tanha, J. (2021). Immunogenicity and humanization of single-domain antibodies. FEBS J.
Rotkiewicz, P., and Skolnick, J. (2008). Fast procedure for reconstruction of full-atom protein models from reduced representations. J. Comput. Chem. 29, 1460-1465.
Salvador, J. P., Vilaplana, L., and Marco, M. P. (2019). Nanobody: outstanding features for diagnostic and therapeutic applications. Anal. Bioanal. Chem. 411, 1703-1713.
Sanner, M. F., Olson, A. J., and Spehner, J. C. (1996). Reduced surface: an efficient way to compute molecular surfaces. Biopolymers 38, 305-320.
Scully, M., Cataland, S. R., Peyvandi, F., Coppo, P., Knobl, P., Kremer Hovinga, J. A., Metjian, A., de la Rubia, J., Pavenski, K., Callewaert, F., et al. (2019). Caplacizumab treatment for acquired thrombotic thrombocytopenia purpura. N. Engl. J. Med. 380, 335-346.
Song, J., Tan, H., Mahmood, K., Law, R. H., Buckle, A. M., Webb, G. I., Akutsu, T., and Whisstock, J. C. (2009). Prodepth: predict residue depth by support vector regression approach from protein sequences only. PLoS One 4, e7072.
Steeland, S., Vandenbroucke, R. E., and Libert, C. (2016). Nanobodies as therapeutics: big opportunities for small antibodies. Drug Discov. Today 21, 1076-1113.
Sun, D., Sang, Z., Kim, Y. J., Xiang, Y., Cohen, T., Belford, A. K., Huet, A., Conway, J. F., Sun, J., Taylor, D. J., et al. (2021). Potent neutralizing nanobodies resist convergent circulating variants of SARS-CoV-2 by targeting diverse and conserved epitopes. Nat. Commun. 12, 4676.
Swindells, M. B., Porter, C. T., Couch, M., Hurst, J., Abhinandan, K R., Nielsen, J. H., Macindoe, G., Hetherington, J., and Martin, A. C. (2017). abYsis: integrated antibody sequence and structure-management, analysis, and prediction. J. Mol. Biol. 429, 356-364.
Vaisman-Mentesh, A., Rosenstein, S., Yavzori, M., Dror, Y., Fudim, E., Ungar, B., Kopylov, U., Picard, O., Kigel, A., Ben-Horin, S., et al. (2019). Molecular landscape of anti-drug antibodies reveals the mechanism of the immune response following treatment with TNFalpha antagonists. Front. Immunol. 10, 2921.
Vanlandschoot, P., Stortelers, C., Beirnaert, E., Ibanez, L. I., Schepens, B., Depla, E., and Saelens, X. (2011). Nanobodies (R): new ammunition to battle viruses. Antivir. Res. 92, 389-407.
Vincke, C., Loris, R., Saerens, D., Martinez-Rodriguez, S., Muyldermans, S., and Conrath, K. (2009). General strategy to humanize a camelid single-domain antibody and identification of a universal humanized nanobody scaffold. J. Biol. Chem. 284, 3273-3284.
Webb, B., and Sali, A. (2016). Comparative protein structure modeling using MODELLER. Curr. Protoc. Bioinf. 54, 561-5637.
Xiang, Y., Nambulli, S., Xiao, Z., Liu, H., Sang, Z., Duprex, W. P., SchneidmanDuhovny, D., Zhang, C., and Shi, Y. (2020). Versatile and multivalent nanobodies efficiently neutralize SARS-CoV-2. Science 370, 1479-1484.
Xiang, Y., Sang, Z., Bitton, L., Xu, J., Liu, Y., Schneidman-Duhovny, D., and Shi, Y. (2021). Integrative proteomics identifies thousands of distinct, multi-epitope, and high-affinity nanobodies. Cell Syst. 12, 220-234 e229.
Yuan, C., Chen, H., and Kihara, D. (2012). Effective inter-residue contact definitions for accurate protein fold recognition. BMC Bioinf. 13, 292.

Yuan, M., Wu, N. C., Zhu, X., Lee, C. D., So, R. T. Y., Lv, H., Mok, C. K. P., and Wilson, I. A. (2020). A highly conserved cryptic epitope in the receptor binding domains of SARS-CoV-2 and SARS-CoV. Science 368, 630-633.
Zhao, W. H., Liu, J., Wang, B. Y., Chen, Y. X., Cao, X. M., Yang, Y., Zhang, Y. L., Wang, F. X., Zhang, P. Y., Lei, B., et al. (2018). A phase 1, open-label study of LCAR-B38M, a chimeric antigen receptor T cell therapy directed against B cell maturation antigen, in patients with relapsed or refractory multiple myeloma. J. Hematol. Oncol. 11, 141.

What is claimed is:

1. A method of humanizing nanobodies comprising:
a) matching nanobody sequences to human variable heavy chain (VHₕᵤₘₐₙ) sequences;
b) performing a sequence alignment of the nanobodies and human variable chain sequences to identify framework sequence differences;
c) performing intramolecular interaction analysis on nanobody structure measuring the structural stability to establish residues that are least likely to change the nanobody structure and more likely to be recognized by the human immune system;
d) performing solvent accessibility analysis measuring to establish residues whose sidechains are exposed to solvent and most likely to be recognized by human immune system; and
e) substituting residues that are not likely to have an impact on the structure, solubility, binding ability, but still exposed and likely to be recognized by human immune system.

2. The method of claim 1, wherein the substitution comprises a substitution at framework region (FR) 1 (FR1) of the nanobody.

3. The method of claim 2, wherein the substitution comprises an alanine to proline substation at residue 14 (A14P) and/or an arginine to phenylalanine substitution at residue 27 (R27F).

4. The method of claim 1, wherein the substitution comprises a substitution at FR2 of the nanobody.

5. The method of claim 4, wherein the substitution comprises a phenylalanine to valine substitution at residue 37 (F37V), tyrosine to valine (Y37V), a glutamate to glycine substitution at residue 44 (E44G), an arginine to leucine substitution at residue 45 (R45L), a phenylalanine to tryptophan substitution at residue 47 (F47W), and/or a leucine to tryptophan substitution at residue 47 (L47W).

6. The method of claim 1, wherein the substitution comprises a substitution at FR3 of the nanobody.

7. The method of claim 6, wherein the substitution comprises a valine to leucine substitution at residue 75 (V75L), a tyrosine to arginine substitution at residue 83 (K83R), a proline to alanine substitution at residue 84 (P84A), an alanine to arginine substitution at residue 94 or an alanine to lysine substitution at residue 94 (A94K).

8. The method of claim 1, wherein the substitution comprises a substitution at FR4 of the nanobody.

9. The method of claim 8, wherein the substitution comprises a glutamine to leucine substitution at residue 108 (Q108L).

10. The method of any of claims 1-9, further comprising performing nanobody structure prediction based on sequence.

11. The method of any of claims 1-10, wherein when nanobody-antigen complex structure is available, the method further comprises performing intermolecular contact analysis on the nanobody-antigen complex structure.

12. The method of any of claims 1-11, wherein measuring structural differences comprises measuring the distance between side chains and the antibody surface; wherein a residue with a distance of 3 Å or less is considered buried; wherein when a residue that is buried on a human antibody and exposed on a nanobody the residue is not substituted to humanize the nanobody.

13. The method of any of claims 1-12, wherein the intramolecular contact analysis measures interactions between FR2 and FR4 and/or interactions between FR2 and complementarity-determining region (CDR) 3 (CDR3).

14. The method of any of claims 1-12, wherein the intramolecular contact analysis measures specific intramolecular disulfide bonds present in nanobodies and not present in human variable heavy chains.

15. The method of any of claims 1-12, wherein the intermolecular contact analysis indicates direct involvement of residues in antigen binding; wherein residues directly involved in antigen binding are not substituted to humanize the nanobody.

16. The method of any of claims 1-15, wherein the process further comprises back translating humanized sequences into DNA sequences and synthesizing the sequence.

17. The method of any of claims 1-16, wherein humanization analysis steps b-d are performed using a system for automated structure guided nanobody humanization comprising
a) nanobody structure modeling;
b) sequence annotation;
c) sequence analysis;
d) structural analysis; and
e) creating a humanization score.

18. The methods of any of claims 1-17 wherein an indication of residue frequency of <10%, solvent exposure of residue, structural integrity as determined by intramolecular interaction analysis, and/or antigen binding as determined by intermolecular interaction analysis indicates a residue is selected for hybridization and wherein an indication of residue frequency of >10%, a buried residue, a lack of structural integrity as determined by intramolecular interaction analysis, and/or a lack of antigen binding as determined by intermolecular interaction analysis indicates a residue is excluded for hybridization.

19. A system for automated structure guided nanobody humanization comprising
a) nanobody structure modeling;
b) sequence annotation;
c) sequence analysis;
d) structural analysis; and
e) creating a humanization score.

20. The system of claim 19, further comprising intramolecular interaction analysis and/or intermolecular interaction analysis.
